(19) United States
(12) Patent Application Publication
(10) Pub. No.: US 2008/0208228 A1
(43) Pub. Date: Aug. 28, 2008

(54) Tongue Cleaning Device Comprising Tongue Cleaning Elements Of Different Materials

(75) Inventor: Wolf-Dieter Mueller, Buehl (DE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GlaxoSmithKline Consumer Healthcare GmbH & Co KG, Buehl (DE)

(21) Appl. No.: 11/813,703

(22) PCT Filed: Jan. 10, 2006
(86) PCT No.: PCT/EP06/00181

§ 371 Date: Jul. 11, 2007

(30) Foreign Application Priority Data
Jan. 12, 2005 (GB) 0500575.6

(51) Int. Cl. A61B 17/24

(52) U.S. Cl. 606/161

(57) ABSTRACT

A tongue-cleaning device comprised of a handle and a head, the head having tongue cleaning elements made of both a relatively harder material and a relatively softer material.

This invention relates to a novel device for tongue cleaning.

It is recognised that regular cleaning of the surface of the tongue is an important part of oral hygiene as deposits on the tongue surface can lead to bad breath. Typically tongue cleaning devices comprise a scraper, typically a ribbed member, which can be drawn across the tongue. Inumerable types of tongue cleaning device are known, for example as disclosed in WO-A-01/45573, U.S. Pat. No. 5,792,159, U.S. Pat. No. 5,984,935, U.S. Pat. No. 5,810,856, U.S. Pat. No. 6,083,235, US-A-2001/0004701 and JP-A-2001161720 among many other publications. A particular type of tongue cleaning device has recently been commercially launched, being a toothbrush sold under the name “Aquafresh Tooth & Tongue” with a set of widthways oriented elastomer ribs on a surface of the head opposite to that from which the bristles extend. This toothbrush is a typical two-component toothbrush comprising a “skeleton” of a first component hard plastic material, and a second component being the elastomer ribs and other elastomer material parts, e.g. elastomer material grip pads.

It is an object of this invention to provide an improved device for tongue cleaning.

According to this invention a tongue-cleaning device is provided comprised of a handle and a head disposed along a head-handle longitudinal direction, the head having a surface on which are situated tongue cleaning elements, wherein the tongue cleaning elements comprise elements made of a relatively harder material and a relatively softer material.

The tongue cleaning elements are suitably ridges of the relatively harder material and relatively softer material aligned with their length direction transverse to the longitudinal direction. Suitably the ridges may have a triangular section with the apex distant from the surface, optionally the sides of the triangle and its apex distant from the surface may be rounded. For example the tongue cleaning elements may have triangular section in which the side of the triangle adjacent to the handle extends perpendicularly to the surface, or encloses an acute angle relative to the surface immediately adjacent to the side. In plan as viewed looking down onto the surface such ridges may be straight, curved or include a non-180° angle, e.g. such that in plan the ridge is of a chevron shape, pointing away from or toward the handle.

The tongue cleaning elements comprise elements made of a relatively harder material and a relatively softer material may be arranged relative to each other in various ways. For example individual elements made of a relatively harder material may alternate longitudinally with individual elements of the relatively softer material. For example plural elements made of a relatively harder material may alternate longitudinally with plural elements of the relatively softer material. For example adjacent a longitudinal end of the head there maybe plural elements being only elements made of the relatively harder material, and adjacent the opposite longitudinal end of the head there may be plural elements being only elements made of the relatively softer material. In one form of this latter arrangement, adjacent to the end of the head furthest from the handle there may be one element made of the relatively softer material, or longitudinally successive plural elements made only of the relatively softer material, and the remainder of the elements toward the opposite end may be elements made only of the relatively harder material.

The head may have two surfaces opposed across the length direction, on one of said surfaces being the tongue cleaning elements comprising elements made of a relatively harder material and a relatively softer material.

The head may have two surfaces opposed across the length direction, on both of said surfaces being the tongue cleaning elements comprising elements made of the relatively harder material and the relatively softer material.

The head may have two surfaces opposed across the length direction, on one of said surfaces being the tongue cleaning elements comprising elements made of a relatively harder material and a relatively softer material, and on the other of said surfaces there being only tongue cleaning elements made of the relatively harder material.

The head may have two surfaces opposed across the length direction, on one of said surfaces being the tongue cleaning elements comprising elements made of a relatively harder material and a relatively softer material, and on the other of said surfaces there being only tongue cleaning elements made of the relatively softer material.

The respective sizes and shapes of the tongue cleaning elements on the two opposed surfaces may be the same or different.

The head may be flattened in shape, having a width dimension perpendicular to the longitudinal direction, and a thickness dimension perpendicular to the longitudinal and width directions, the thickness direction being less than the width dimension. Typically the thickness dimension may be 10-20% of the maximum width dimension. The head may be of a shape which increases in width dimension with distance from the handle, e.g. being of a rounded isosceles triangular shape with its apex adjacent the handle.

The relatively harder material is preferably a plastics material, typically of the type commonly used for making toothbrushes, e.g. a polypropylene, an ABS material, or a polyamide. The relatively softer material is preferably also a plastics material, for example a flexible material such as a polyethylene or a thermoplastic elastomer e.g. a SEBS material, e.g. such as Santoprene, typically of the type commonly used for making so called two-component toothbrushes, e.g. with a harder plastics material skeleton and thermoplastic elastomer parts e.g. to enhance grip. For example the above-mentioned “Aquafresh Tooth & Tongue” incorporates such relatively harder and softer materials.

The handle and head may be integral, i.e. made as a single piece. There may be a flexible link between the head and handle. A particular type of flexible link comprises a composite region comprising parts made of the relatively harder and softer materials, typically in the form of a link of the relatively harder material between the head and handle, of dimensions such that the link is flexible, enclosed in a mass of the relatively softer material.

The device of the invention may be made in a generally conventional manner by firstly making the relatively harder material part(s) of the device, typically by injection moulding, then attaching the relatively softer material part(s), typically by enclosing the relatively harder material part(s) of the device in the cavity of an injection mould defining the shape of the relatively softer material part(s), then injecting the relatively softer material into the mould to thereby form the relatively softer material part(s). This process is analogous to the processes used for making known two component toothbrushes mentioned above.

The invention will now be described by way of example only with reference to the accompanying drawings.

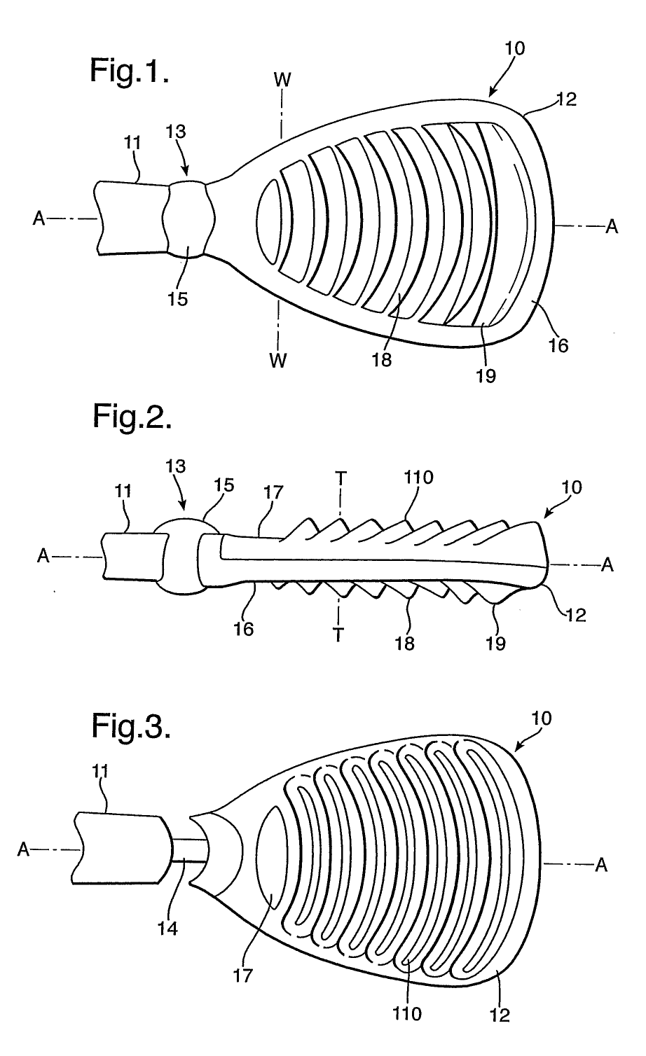

FIG. 1 shows a plan view of a surface of the head and adjacent part of the handle.

FIG. 2 shows a side view of the head and adjacent part of the handle.

FIG. 3 shows a plan view of the opposite surface of the head and adjacent part of the handle.

10 tongue cleaning device

11 handle

12 head

13 flexible link

14 thin integral link

15 spherical mass of thermoplastic elastomer

16,17 surfaces opposed across the length direction.

18 tongue cleaning elements of hard plastic material.

19 tongue cleaning elements of thermoplastic elastomer material.

110 tongue cleaning elements of thermoplastic elastomer material

Referring to FIGS. 1, 2 and 3 a tongue-cleaning device is shown overall 10 comprised of a handle (part shown) 11 and a head 12 disposed along a head-handle longitudinal direction A-A. FIG. 3 is part cut away.

The handle 11 and head 12 are integrally made of a relatively hard plastic material such as a grade of polypropylene routinely used for making toothbrushes. There is a flexible link 13 between the head 11 and handle 12. This flexible link 13 comprises a composite region comprising a thin integral link 14 of the relatively harder material between the head 11 and handle 12 being of dimensions such that the link is flexible, enclosed in a spherical mass 15 of a thermoplastic elastomer such as that routinely used for the soft grip pads of toothbrushes. Such a link is analogous to the construction of link used between the heads and handles of known types of toothbrushes.

The head 11 is flattened in shape, having a width dimension W-W perpendicular to the longitudinal direction A-A, and a thickness dimension T-T perpendicular to the longitudinal and width directions, the thickness direction being less than the width dimension. As seen in the plan views of FIGS. 1 and 3 the head is in plan a rounded isosceles triangular shape with its apex adjacent the handle. The head has two surfaces 16, 17 opposed across the length direction.

On the surface 16 are situated tongue cleaning elements 18,19. These comprise elements 18 which are made integrally with head 11, of the hard plastic material of the head, and elements 19 which are made of a thermoplastic elastomer material, which is softer than the hard plastic material of elements 18.

The tongue cleaning elements 18,19 are in the form of ridges aligned with their length direction transverse to the longitudinal direction A-A. As is seen in the side view FIG. 2 the ridges 18,19 have a triangular section with their apex distant from the surface. In plan as seen in FIGS. 1 and 3 looking down onto the surface 16 the ridges 18,19 are curved with their convex side away from the handle. Adjacent to the end of the head furthest from the handle there are plural, two are shown but there may be one or more than two, longitudinally sequential elements 19 made of the thermoplastic elastomer material. The remainder of the elements 18 toward the opposite end of the head, specifically six are shown but there may be more or less, are elements 19 made only of the relatively harder plastic material of the head 12.

On the opposite surface 17 are situated tongue cleaning elements 110, which are made of the thermoplastic elastomer material which is softer than the hard plastic material of elements 18. There are no tongue cleaning elements on surface 17 made of the plastic material of the head 11.

The tongue cleaning elements 110 are in the form of ridges aligned with their length direction transverse to the longitudinal direction A-A. As is seen in the side view FIG. 2 the ridges 110 have a triangular section with their apex distant from the surface. In plan as seen in FIG. 3 looking down onto the surface 17 the ridges 110 are curved with their convex side away from the handle.

The device 10 has been made in a generally conventional manner by firstly making the relatively harder material part(s) of the device, typically by injection moulding, then attaching the relatively softer material part(s), typically by enclosing the relatively harder material part(s) of the device in the cavity of an injection mould defining the shape of the relatively softer material part(s), then injecting the relatively softer material into the mould to thereby form the relatively softer material part(s). This process is analogous to the processes used for making known two component toothbrushes mentioned above.

1. A tongue-cleaning device comprised of a handle and a head disposed along a head-handle longitudinal direction, the head having a surface on which are situated tongue cleaning elements, wherein the tongue cleaning elements comprise elements made of a relatively harder material and a relatively softer material.

2. A tongue-cleaning device according to claim 1 wherein the tongue cleaning elements are ridges of the relatively harder material and relatively softer material, aligned with the length direction of the ridges transverse to the longitudinal direction.

3. A tongue-cleaning device according to claim 2 wherein the ridges have a triangular section with the apex distant from the surface.

4. A tongue-cleaning device according to claim 1 wherein individual tongue-cleaning elements made of a relatively harder material alternate longitudinally with individual elements of the relatively softer material.

5. A tongue-cleaning device according to claim 1 wherein plural tongue-cleaning elements made of a relatively harder material alternate longitudinally with plural elements of the relatively softer material.

6. A tongue-cleaning device according to claim 1 wherein adjacent a longitudinal end of the head there are plural tongue-cleaning elements being only elements made of the relatively harder material, and adjacent the opposite longitudinal end of the head there are plural tongue-cleaning elements being only elements made of the relatively softer material.

7. A tongue-cleaning device according to claim 6 wherein adjacent to the end of the head furthest from the handle there is one tongue-cleaning element made of the relatively softer material, or longitudinally successive plural tongue-cleaning elements made only of the relatively softer material, and the remainder of the tongue-cleaning elements toward the opposite end of the head closest to the handle are tongue cleaning elements made only of the relatively harder material.

8. A tongue-cleaning device according to claim 1 wherein the head has two surfaces opposed across the length direction, on one of said surfaces being the tongue cleaning elements comprising elements made of a relatively harder material and a relatively softer material.

9. A tongue-cleaning device according to claim 8 wherein on one of said surfaces are the tongue cleaning elements comprising elements made of a relatively harder material and a relatively softer material, and on the other of said surfaces there are only tongue cleaning elements made of the relatively harder material.

10. A tongue-cleaning device according to claim 1 wherein the head has two surfaces opposed across the length direction, and on both of said surfaces are tongue cleaning elements comprising elements made of the relatively harder material and the relatively softer material.

11. A tongue-cleaning device according to claim 1 wherein the head has two surfaces opposed across the length direction, on one of said surfaces are the tongue cleaning elements comprising elements made of a relatively harder material and a relatively softer material, and on the other of said surfaces there are only tongue cleaning elements made of the relatively softer material.

12. A tongue-cleaning device according to claim 1 wherein the head is flattened in shape, having a width dimension perpendicular to the longitudinal direction, and a thickness dimension perpendicular to the longitudinal and width directions, the thickness direction being less than the width dimension.

13. A tongue-cleaning device according to claim 1 wherein the head has a shape which increases in width dimension with distance from the handle.

14. A tongue-cleaning device according to claim 1 wherein the relatively harder material is a plastics material, and the relatively softer material is also a plastics material.

15. A tongue-cleaning device according to claim 14 wherein the relatively softer material is a polyethylene or thermoplastic elastomer.

16. A tongue-cleaning device according to claim 1 wherein there is a flexible link between the head and handle.
